(19) United States
(12) Patent Application Publication
(10) Pub. No.: US 2020/0085625 A1
(43) Pub. Date: Mar. 19, 2020

(54) ABSORBENT BIOPHOTONIC DEVICES AND SYSTEMS FOR WOUND HEALING

(71) Applicant: KLOX TECHNOLOGIES INC., Laval (CA)

(72) Inventors: Carlo BELLINI, Mont-Royal (CA); Nikolaos LOUPIS, Athens (GR); Remigio PIERGALLINI, San Benedetto del Tronto (IT); David OHAYON, Dollard-des-Ormeaux (CA)

(21) Appl. No.: 16/610,568

(22) PCT Filed: May 4, 2018
(86) PCT No.: PCT/CA2018/050533

§ 371 Date: Nov. 4, 2019

Related U.S. Application Data
(60) Provisional application No. 62/501,721, filed on May 4, 2017.

(51) Int. Cl. A61F 13/00; A61N 5/06

(52) U.S. Cl. CPC A61F 13/00063; A61F 13/00008; A61F 13/00029; A61N 2005/0662; A61F 13/00068; A61N 2005/0645; A61N 5/062

(57) ABSTRACT

The present technology generally relates to products, devices, methods and systems for healing of wounds or for wound-treatment therapies. More particularly, but not by way of limitation, the present technology relates an absorbent product impregnated with photoactivatable agents, or light-absorbing molecules, as photoactivatable inserts as wound dressing that can be used together as part of negative-pressure wound therapy (NPWT) system or separately and without the NPWT system.

### CROSS-REFERENCE TO RELATED APPLICATION

This application claims the benefit of and priority to U.S. provisional patent application No. 62/501,721, filed on May 4, 2017, the content of which is herein incorporated in this entirety by reference.

### FIELD OF TECHNOLOGY

The present technology generally relates to products, devices, methods and systems for the healing of wounds. The present technology also relates to products and devices that may be used as part of negative-pressure wound therapy (NPWT) systems.

### BACKGROUND OF TECHNOLOGY

Wound closure involves the inward migration of epithelial and subcutaneous tissue adjacent to the wound. Without sufficient blood flow, the epithelial and subcutaneous tissues surrounding the wound not only receive diminished oxygen and nutrients, but are also less able to successfully fight bacterial infection and thus are less able to naturally close the wound. Until recently, such difficult wounds were addressed only through the use of sutures or staples. Although still widely practiced and often effective, such mechanical closure techniques suffer a major disadvantage in that they produce tension on the skin tissue adjacent the wound. Examples of wounds not readily treatable with staples or suturing include large, deep, open wounds; decubitus ulcers; ulcers resulting from chronic osteomyelitis; and partial thickness burns that subsequently develop into full thickness burns.

As a result of these and other shortcomings of mechanical closure devices, methods and apparatus for draining wounds by applying continuous negative pressures have been developed. When applied over a sufficient area of the wound, such negative pressure has been found to promote the migration toward the wound of epithelial and subcutaneous tissues. In practice, NPWT system, also referred to vacuum assisted closure (VAC) therapy, typically involves the mechanical-like contraction of the wound with simultaneous removal of excess fluid. In this manner, NPWT therapy augments the body's natural inflammatory process while alleviating many of the known intrinsic side effects, such as the production of edema caused by increased blood flow absent the necessary vascular structure for proper venous return.

Because NPWT therapy dictates an atmospherically sealed wound site, the therapy must often be performed to the exclusion of other beneficial, and therefore desirable, wound treatments. One such excluded wound treatment is phototherapy. Phototherapy of wounds relates to methods for wound treatment wherein appropriate wavelengths of light are directed into or about the wound. Typically, phototherapy has been regarded as impracticable in combination with NPWT therapy due to the utilization of opaque materials in the administration of NPWT therapy. Phototherapy would nonetheless be desirable in combination with NPWT.

It is thus an object of the present technology to provide phototherapeutic products, devices and systems that may be readily adaptable and suitable for use in combination with NPWT in, for example, the healing of wounds and/or the treatment of wounds.

### SUMMARY OF DISCLOSURE

According to various aspects, the present technology relates to an absorbent biophotonic device comprising: a photoactivatable core; and at least one absorbent liner disposed on at least a part of the photoactivatable core; wherein the photoactivatable core is photoactivated upon exposure to light to emit fluorescence.

According to various aspects, the present technology relates to an absorbent biophotonic device for treatment and/or healing of a wound, the absorbent biophotonic device comprising: a photoactivatable core; at least one absorbent liner disposed on at least a part the photoactivatable core; wherein the photoactivatable core is photoactivated upon exposure to light to emit fluorescence.

According to various aspects, the present technology relates to the use of the absorbent biophotonic device as defined herein for healing of a wound.

According to various aspects, the present technology relates to the use of the absorbent biophotonic device as defined herein in combination with negative pressure wound therapy (NPWT) for healing of a wound.

According to various aspects, the present technology relates to the use of the absorbent biophotonic device as defined herein in combination with a fluid-permeable film for healing of a wound.

According to various aspects, the present technology relates to the use of the absorbent biophotonic device as defined herein in combination with a fluid-permeable film, a vacuum source and a light source for healing of a wound.

According to various aspects, the present technology relates to an absorbent biophotonic system for healing of a wound on a subject, the absorbent biophotonic system comprising: an absorbent biophotonic device; and a fluid-impermeable film; wherein the absorbent biophotonic device is suitable for placement on a wound and the fluid-impermeable film is suitable for placement over the absorbent biophotonic device and suitable to create an air-tight connection with skin surrounding the wound.

According to various aspects, the present technology relates to a method for wound healing, the method comprising: applying an absorbent biophotonic device as defined herein on a wound; and exposing the absorbent biophotonic device to actinic light for a time sufficient to achieve photoactivation of the photoactivatable core.

According to various aspects, the present technology relates to a method for wound healing, the method comprising: applying an absorbent biophotonic device as defined herein onto a wound; applying a fluid-impermeable film onto the absorbent biophotonic device and surrounding skin of the wound; creating a negative pressure environment between the fluid-impermeable film and the surrounding skin of the wound; and photoactivating the photoactivatable core for a time sufficient to achieve photoactivation of the light-accepting molecules in the photoactivatable core.

According to other aspects, the present technology relates to a method for reducing scarring resulting from surgical wounds.

Other aspects and features of the present technology will become apparent to those ordinarily skilled in the art upon review of the following description of specific embodiments in conjunction with the accompanying drawings.

### BRIEF DESCRIPTION OF DRAWINGS

A detailed description of embodiments of the present disclosure is provided below, by way of example only, with reference to the accompanying drawings, in which:

FIG. 1 is a schematic representation of a cross-sectional view of an absorbent biophotonic device according to one embodiment of the present disclosure.

FIG. 2 is a schematic representation of a cross-sectional view of an absorbent biophotonic device according to one embodiment of the present disclosure, wherein the absorbent biophotonic device is punctured throughout.

FIG. 3 is a schematic representation of an absorbent biophotonic system according to one embodiment of the present technology placed onto a wound located on the forearm of a subject.

FIG. 4 is a graph showing the properties of the light emitted by a photo-stimulated absorbent biophotonic system according to one embodiment of the present disclosure wherein the absorbent biophotonic device of the system comprises a 3 mm-thick polyurethane foam.

FIG. 5 is a graph showing the properties of the light emitted by a photo-stimulated absorbent biophotonic system according to one embodiment of the present disclosure wherein the absorbent biophotonic device of the system comprises a 9 mm-thick polyurethane foam.

FIG. 6 is a graph showing the properties of the light emitted by a photo-stimulated absorbent biophotonic system according to one embodiment of the present disclosure wherein the absorbent biophotonic system is used with a negative pressure system (Vacuum: −50 mmHg).

FIG. 7 is a graph showing the properties of the light emitted by another photo-stimulated absorbent biophotonic system according to one embodiment of the present disclosure wherein the absorbent biophotonic system is used with a negative pressure system (Vacuum: −100 mmHg).

FIG. 8 is a graph showing the properties of the light emitted by a further absorbent biophotonic system according to one embodiment of the present disclosure wherein the absorbent biophotonic system is used with a negative pressure system (Vacuum: −200 mmHg).

FIG. 9 is a graph showing the properties of the light emitted by an absorbent biophotonic system according to one embodiment of the present disclosure.

FIGS. 10A-10C are schematic representations of a cross-sectional view of an absorbent biophotonic system according to various embodiments of the present disclosure, wherein the light source is located within the absorbent biophotonic device of the system.

It is to be expressly understood that the description and drawings are only for the purpose of illustrating certain embodiments of the present disclosure and are an aid for understanding. They are not intended to be a definition of the limits of the disclosure and/or of the technology.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant technology. Hence, the following specification is intended to illustrate some particular embodiments of the technology, and not to exhaustively specify all permutations, combinations and variations thereof.

As used herein, the singular form “a,” “an” and “the” include plural referents unless the context clearly dictates otherwise.

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It will be understood that when an element is referred to as being “on” another element, it can be directly on the other element or intervening elements may be present therebetween.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section.

Relative terms, such as “lower” or “bottom”, “upper” or “top”, “left” or “right”, “above” or “below”, “front” or “rear” may be used herein to describe one element's relationship to another element as illustrated in the Figures. It will be understood that relative terms are intended to encompass different orientations of the device in addition to the orientation depicted in the Figures.

As used herein, the term “biophotonic” means the generation, manipulation, detection and application of photons in a biologically relevant context. In other words, biophotonic compositions exert their physiological effects primarily due to the generation and manipulation of photons. As used herein, the expression “biophotonic composition” refers to a composition as described herein that may be activated by light to produce photons for biologically relevant applications.

As used herein the expression “fluorescence biomodulation” refers to a form of photobiomodulation which utilizes fluorescence energy to induce multiple transduction pathways that can modulate biological processes through the activation of photoacceptors found within many different cell and tissue types. Photoacceptors are molecules (i.e., light-absorbing molecules) that do not explicitly specialize in light absorption but possess the ability to do so in the presence of light which in turn can enhance their functioning. To generate fluorescence, light-absorbing molecules are employed to translate light energy into a high-energy emission of fluorescence through a mechanism known as stokes shift. Fluorescence biomodulation may differ from photobiomodulation in that it employs fluorescence as a photo vehicle to induce biomodulation. Fluorescence, as generated by light-absorbing molecules, is displayed as a broad spectral distribution of wavelengths and/or frequencies which can be controlled to penetrate tissues to various degrees. Tailoring fluorescence biomodulation allows compatibility between the specific emissions of fluorescence and the unique light absorbing characteristics of different cell and tissue types. Shorter wavelengths (<600 nm) within the visible spectrum cannot penetrate deep into tissue and are localized within the epidermis or dermis. Conversely, longer wavelengths (>600 nm) within the visible spectrum penetrate further up into the hypodermis.

The term “topical” as used herein means as applied to body surfaces, such as the skin, mucous membranes, vagina, oral cavity, internal surgical wound sites, and the like.

Terms and expressions “light-absorbing molecule”, “photoactivating agent”, “photoactivator”, “photoacceptors” and “chromophore” are used herein interchangeably. A light-absorbing molecule means a compound, when contacted by light irradiation, is capable of absorbing the light. The light-absorbing molecule readily undergoes photoexcitation and can then transfer its energy to other molecules or emit it as light. The term light-absorbing molecule may be interchangeable with the term light-accepting molecule where both terms represent the same meaning.

As used herein, the term “wound” means an injury to any tissue, including for example, acute, subacute, and non-healing wounds. Examples of wounds may include both open and closed wounds. Wounds include, for example, skin diseases that result in a break of the skin or in a wound, clinically infected wounds, burns, incisions, excisions, lesions, lacerations, abrasions, puncture or penetrating wounds, gunshot wounds, surgical wounds, contusions, hematomas, crushing injuries, ulcers, scarring (cosmesis), wounds caused by periodontitis. As used herein, the expression “non-healing wounds” means wounds that do not heal in an orderly set of stages and a predictable amount of time and rate in the way that most normally-healing wounds heal, and non-healing wounds include, but are not limited to: incompletely healed wounds, delayed healing wounds, impaired wounds, difficult to heal wounds and chronic wounds. Examples of such non-healing wounds include diabetic foot ulcers, vascultic ulcers, pressure ulcers, decubitus ulcers, infectious ulcers, trauma-induced ulcers, burn ulcers, ulcerations associated with pyoderma gangrenosum, dehiscent and mixed ulcers. A non-healing wound may include, for example, a wound that is characterized at least in part by: 1) a prolonged inflammatory phase; 2) a slow forming extracellular matrix; and/or 3) a decreased rate of epithelialization or closure. As used herein, the expression “chronic wound” means a wound that has not healed within about 4 to 6 weeks. Chronic wounds include venous ulcers, venous stasis ulcers, arterial ulcers, pressure ulcers, diabeteic ulcers, and diabetic foot ulcers.

In one embodiment, the present technology relates to an absorbent biophotonic device which is capable of emitting fluorescence when activated by light.

In some embodiments, the absorbent biophotonic device of the present technology may be used in the treatment or healing of wounds. In some other embodiments, the absorbent biophotonic device of the present technology may be used to promote closure of a wound.

In some embodiments, the absorbent biophotonic device of the present technology is used in combination with a fluid-impermeable film to form an absorbent biophotonic system that may be used in the treatment of wounds and/or that may be used to promote closure of a wound.

In some embodiments, the absorbent biophotonic device of the present technology is used in combination with a fluid-impermeable film and a vacuum system to form an absorbent biophotonic system that may be used in the treatment of wounds and/or that may be used to promote closure of a wound.

In some embodiments, the absorbent biophotonic device of the present technology is used in combination with a fluid-impermeable film, a vacuum system and a light source to form an absorbent biophotonic system that may be used in the treatment of wounds and/or that may be used to promote closure of a wound.

i) Absorbent Biophotonic Device

In some embodiments, the absorbent biophotonic device of the present disclosure comprises a photoactivatable core and an absorbent material.

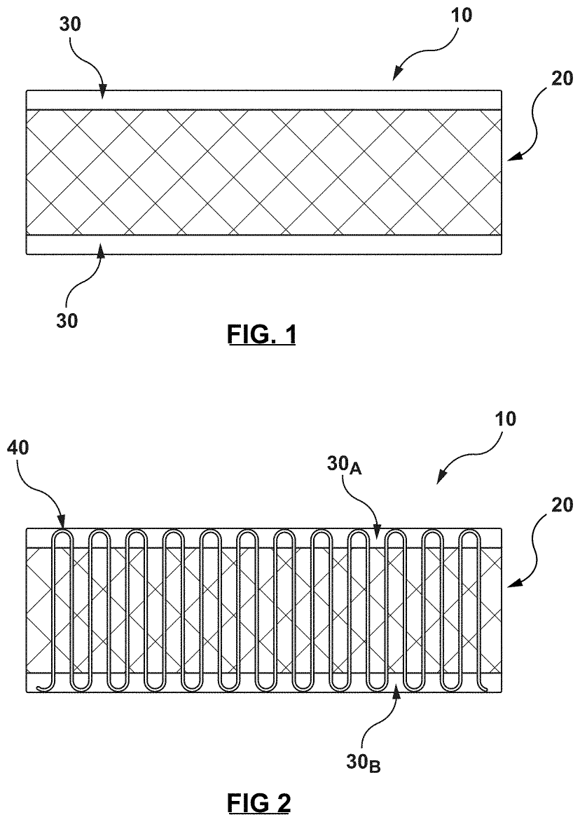

FIG. 1 illustrates an absorbent biophotonic device according to one embodiment of the present technology wherein the absorbent biophotonic device (10) is an absorbent biophotonic pad. The absorbent biophotonic pad (10) comprises a photoactivatable core (20) and an absorbent liner (30). In this embodiment, the absorbent biophotonic pad (10) comprises two layers of absorbent liner (30A, 30B) and the photoactivatable core (20) is placed (i.e., sandwiched) between the two layers of absorbent liners: a top layer of absorbent liner (30A) and a bottom layer of absorbent liner (30B). The bottom absorbent liner (30B) is placed in direct contact with the wound. In some other embodiments, the absorbent biophotonic pad (10) comprises only one layer of absorbent liner (30) (not shown), which may act as a top absorbent liner or a bottom absorbent liner. The photoactivatable core (20) and/or the absorbent liner (30) are made from absorbent materials as will be described below. The photoactivatable core (20) comprises light-accepting molecules.

In some instances, the absorbent liner (30) is connected to the photoactivatable core (20) by a suitable connection means (not shown). Examples of suitable connection means include, but are not limited to, adhesives (e.g., glue, fixative, gum, paste, or the like), which is applied to one or the two surfaces that are to be connected one to; and stitches as discussed below. Other techniques which are known in the art may be used to connect the absorbent liner (30) to the photoactivatable core (20).

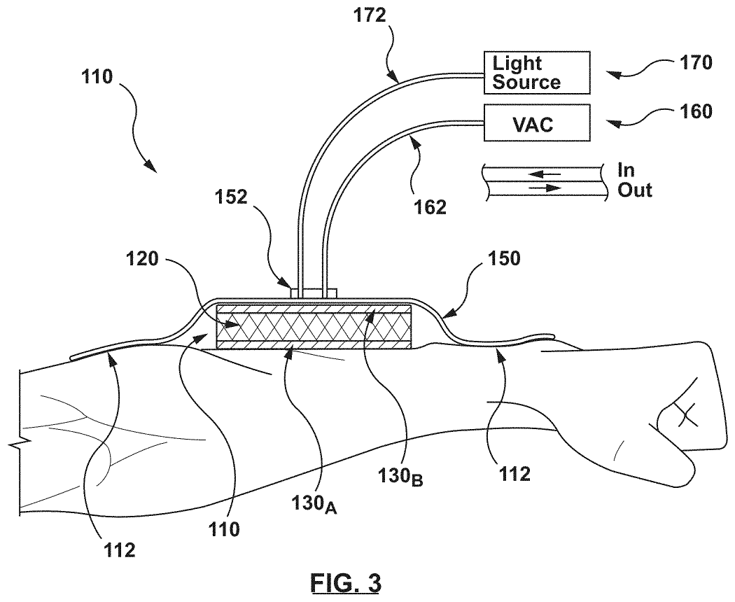

As illustrated in FIG. 2, the photoactivatable core (20) and an absorbent liner (30) are held together through a plurality of needle punctures (40). The needle punctures (40) create openings or channels in the absorbent biophotonic pad (10) which, besides holding the photoactivatable core (20) and the layers of absorbent liner (30A, 30B) together, may allow light shed onto the absorbent biophotonic pad (10) to access the inner core of the absorbent biophotonic pad (10) such as to photoactivate the light-accepting molecules located therein as will be discussed below. In some instances, the openings or channels serve as connection means to connect the photoactivatable core (20) and the absorbent material (30) together. In some instances, the opening or channels created by the punctures allow light being shed onto the absorbent biophotonic device (10) to access the inner core of the absorbent biophotonic device (10) such as to photoactivate the photoactivatable agents located therein.

The different components of the absorbent biophotonic device (10) according to several embodiments of the present technology will now be discussed in greater details.

ii) Absorbent Liner

In some embodiments, the absorbent liner (30) absorbs fluids released from the wound to be treated. In some other instances, the absorbent liner (30) also prevents or decreases leaching of the light-accepting molecules out of the photoactivatable core (20).

In some implementations, the absorbent liners 30A and 30B each have a thickness of about 4.0 cm, about 3.75 cm, about 3.5 cm, about 3.25 cm, about 3.0 cm, about 2.75 cm, about 2.5 cm, about 2.25 cm, about 2.0 cm, about 1.75 cm, about 1.5 cm, about 1.25 cm, about 1.0 cm, about 0.75 cm, about 0.5 cm, or about 0.25 cm. In some instances, the absorbent liners 30A and 30B may have a different thickness.

In some implementations, the absorbent liner (30) of the absorbent biophotonic device (10) is made of a foam material. The foam material useful in the present technology may be made from any suitable type of foam material, including open celled foam, hydrophilic foam, polyvinyl foams such as white polyvinyl alcohol foam, reticulated foam, or any combination of the foregoing. For example, the foam material can comprise multiple layers of foam, wherein each layer can be made from any of the foregoing materials: A) Fibers B) any number of polymers, and C) any number of configurations.

In some implementations of these embodiments, the foam material is NPWT foam. The NPWT foam preferably has an open porous structure, to allow transmission of the negative pressure to the wound bed, and sufficient mechanical strength to prevent the negative pressure from collapsing the structure of the foam. The levels of pressure vary between about −125 mmHg and about −75 mmHg. In some instances however, the levels of pressure may be greater than −125 mmHg (e.g., between about or lower than −75 mmHg.

Suitable foams that may be used as part of the absorbent liner (30) include polyurethane foam. In some instances, the polyurethane foam is reticulated polyurethane foam having a free internal volume of for examples at least about 80%, or at least about 85%, or at least about 90%, or at least about 95% or higher. In some instances, the polyurethane foam is reticulated polyurethane foam having a free internal volume of no less than 80%. In some instances, the foam has porosities lower than about 30 ppi. In some other instances, the foam has porosities greater than 60 ppi. In some instances, the mean pore diameter is lower than 300 μm. In some other instances, the mean pore diameter is greater than 800 μm. In some instances, the foam has porosities in the range of between about 30 ppi and about 60 ppi (pores per inch) and mean pore diameters in the range of between about 300 μm and about 800 μm.

Other suitable foams include polyethylene foam. In some instances, the polyethylene foam is reticulated polyethylene foam having a free internal volume of for examples at least about 80%, or at least about 85%, or at least about 90%, or at least about 95% or higher. In some instances, the polyethylene foam is reticulated polyethylene foam having a free internal volume of no less than 80%. In some instances, the foam has porosities lower than about 30 ppi. In some other instances, the foam has porosities greater than 60 ppi. In some instances, the mean pore diameter is lower than 300 μm. In some other instances, the mean pore diameter is greater than 800 μm. In some instances, the foam has porosities in the range of between about 30 ppi and about 60 ppi (pores per inch) and mean pore diameters in the range of between about 300 μm and about 800 μm.

Other suitable foams include polypropylene foam. In some instances, the polypropylene foam is reticulated polypropylene foam having a free internal volume of for examples at least about 80%, or at least about 85%, or at least about 90%, or at least about 95% or higher. In some instances, the polypropylene foam is reticulated polypropylene foam having a free internal volume of no less than 80%. In some instances, the foam has porosities lower than about 30 ppi. In some other instances, the foam has porosities greater than 60 ppi. In some instances, the mean pore diameter is lower than 300 μm. In some other instances, the mean pore diameter is greater than 800 μm. In some instances, the foam has porosities in the range of between about 30 ppi and about 60 ppi (pores per inch) and mean pore diameters in the range of between about 300 μm and about 800 μm.

In some implementations, the foam material comprises a plurality of slits. As used herein, the term slit is intended to mean a cut which is generally long and thin, and preferably straight and linear. In practice, slits in foam are typically effectively 2-dimensional as the resilience of the foam means that the slit is essentially closed unless the material is stretched. Suitably the slits are from between about 10 mm and about 70 mm in length, or from between about 20 mm and about 50 mm, or from between about 25 mm and about 40 mm. In some instances, providing slits may confer macroscopic flexibility, while not substantially affecting the microscopic mechanical properties of the body (i.e., to resist compression under negative pressure). This flexibility allows the body of porous material to drape more easily (i.e., to conform to the shape of the wound to be dressed).

It will be clear to the person skilled in the art that the size of the gap between the slits, and the spacing between adjacent series, must be sufficient such that the material retains structural integrity sufficient for it to be handled, used in therapy and removed thereafter without breaking up. If the gaps and spacing were too small, the body would be too weak to achieve this. On the other hand, there is a desire to maximise the size and density of the slits to maximise drapeability. It is preferred that the minimum size of the gaps and/or spacing should certainly be no less than the average pore diameter. It is more preferred that gaps and/or spacing are at least 5 times the average pore diameter; given that the average pore diameter for the foam material is in the range of between about 300 μm and about 800 μm, this gives a gap or spacing of between about 1500 μm and about 4000 μm. A spacing between adjacent linear series of slits should be kept reasonably small to provide the desired amount of flexibility and hence drapeability. The spacing may not be more than 50 times the average pore diameter (typically from 15 mm to 40 mm depending on pore density), or not more than 30 times than average pore diameter (typically from 9 mm to 24 mm depending on pore density).

Other suitable foams are known in the art and may be equally employed in the composition of the absorbent liner (30) of the present technology.

iii) Photoactivatable Core

In some embodiments, the photoactivatable core (20) of the absorbent biophotonic device (10) is made from photoactivatable fibers. As used herein, the term “fiber” relates to a string or a thread or a filament used as a component of composite materials. Fibers may be used in the manufacture of other materials such as for example, but not limited to, fabrics. The photoactivatable fibers are, but not limited to, synthetic fibers, natural fibers, and textile fibers. Synthetic fibers may be made from a polymer or a combination of different polymers. In some instances, the polymer is a thermoplastic polymer.

In some instances, the polymer is acrylic, acrylonitrile butadiene styrene (ABS), polybenzimidazole (PBI), polycarbonate, polyether sulfone (PES), polyetherether ketone (PEEK), polyetherimide (PEI), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayons, poly(methyl methacrylate) (PMMA), or from any mixture thereof. In some other instances, the fibers may be made from glycolic acid, copolymer lactide/glycolide, polyester polymer, copolymer polyglycolic acid/trimethylene carbonate, natural protein fiber, cellulose fiber, polyamide polymer, polymer of polypropylene, polymer of polyethylene, nylon, polymer of polylactic acid, polymer of polybutylene terephthalate, polyester, copolymer polyglycol, polybutylene, polymer of poly methyl methacrylate, or from any mixture thereof.

In some implementations, the fibers of the present disclosure may be coextruded fibers that have two distinct polymers forming the fiber, usually as a core-sheath or side-by-side. The fibers may be coated with silicone.

In some implementations, the fibers may be composed of a single strand(mono-filament) or may be composed of a plurality of strands (multi-filaments). The photoactivatable fibers that are multifilament may also be intertwined or braided or twisted (i.e., the multifilaments are intertwined, braided or twisted to form the fibers).

In some implementations, the diameter of the photoactivatable fiber define herein (taken individually, monofilament) varies between about 15 microns and about 500 microns, between about 25 microns and about 500 microns, between about 50 microns and 400 microns, between about 50 microns and about 300 microns, preferably between about 50 microns and about 250 microns, preferably between about 75 microns and about 300 microns, and most preferably between about 75 microns and about 250 microns. In some specific implementations, the diameter of the photoactivatable fibers defined herein is about 15 microns, about 20 microns, about 25 microns, about 50 microns, about 75 microns, about 100 microns, about 125 microns, about 150 microns, about 175 microns, about 200 microns, about 225 microns, about 250 microns, about 250 microns, about 275 microns, about 300 microns, about 325 microns, about 350 microns, about 375 microns, about 400 microns, about 425 microns, about 450 microns, about 475 microns, about 500 microns. In some instances, the diameter of the photoactivatable fibers defined herein (taken individually) is about 31 microns.

In some implementations, the photoactivatable fibers defined herein show a medium to high resistance to mechanical pulling and stretching forces. In some implementations, the photoactivatable fibers defined here are resilient and have the ability to stretch and to reform to their original size and shape.

In some implementations, the photoactivatable fibers have a linear mass density of between about 300 Deniers and about 500 Deniers, or between about 300 and about 480 Deniers. As used herein, the term “Denier” refers to a unit of measure for the linear mass density of fibers, is defined as the mass in grams per 9000 meters.

Examples of photoactivatable fibers have been described in WO 2016/065488, which is incorporated herein in its entirety.

In some embodiments, the photoactivatable core (20) comprises light-accepting molecules. In some implementations of these embodiments, the light-accepting molecules are comprised in and/or on the photoactivatable fibers of the photoactivatable core (20).

In some implementations of these embodiments, the photoactivatable agents may be present on the surface of the photoactivatable fibers. In some other implementations, the photoactivatable agents may be embedded into the photoactivatable fibers. In other implementations, a portion of the photoactivatable agents may be present on the surface of the photoactivatable fibers, whereas another portion of the photoactivatable agents may be embedded into the photoactivatable fibers.

In some implementations, the photoactivatable agent is a chemical compound which, when exposed to the light is photoexcited and can then transfer its energy to other molecules or emit it as light, such as for example fluorescence. For example, in some instances, the photoactivable agent when photoexcited by the light may transfer its energy to enhance or accelerate light dispersion or to other molecules. Examples of photoactivable agents include, but are not limited to light-absorbing molecules (also known as fluorescent compounds (or stains)). Other dye groups or dyes (biological and histological dyes, food colorings, carotenoids, and other dyes) can also be used. Suitable photoactivatable agent can be those that are Generally Regarded As Safe (GRAS).

In certain implementations, the photoactivatable fibers of the present disclosure comprise a first photoactivatable agent. In some implementations, the first photoactivatable agent absorbs at a wavelength in the range of the visible spectrum, such as at a wavelength of about 380 nm to about 800 nm, about 380 nm to about 700, about 400 nm to about 800, or about 380 nm to about 600 nm. In other embodiments, the first photoactivating agent absorbs at a wavelength of about 200 nm to about 800 nm, of about 200 nm to about 700 nm, of about 200 nm to about 600 nm or of about 200 nm to about 500 nm. In one embodiment, the first photoactivatable agent absorbs at a wavelength of about 200 nm to about 600 nm. In some embodiments, the first photoactivatable agent absorbs light at a wavelength of about 200 nm to about 300 nm, of about 250 nm to about 350 nm, of about 300 nm to about 400 nm, of about 350 nm to about 450 nm, of about 400 nm to about 500 nm, of about 450 nm to about 650 nm, of about 600 nm to about 700 nm, of about 650 nm to about 750 nm or of about 700 nm to about 800 nm.

In some implementations, the photoactivatable agents emit light within the range of about 400 nm and about 800 nm.

In some implementations of these embodiments, the photoactivatable agent is a xanthene dye.

In some implementations of these embodiments, the photoactivatable agent is Eosin (Eosin Y, derivatives of Eosin Y, Eosin B, or derivatives of Eosin B or any combination thereof) or Erythrosin or Fluorescein or Rose Bengal or any combination thereof.

In the implementations wherein the photoactivatable fibers have the photoactivatable agent on their surface (i.e., the surface of the fibers that is in contact with the surrounding environment of the fiber), such photoactivatable fibers may be prepared by being dipped into a photoactivatable agent composition comprising one or more photoactivatable agents and a carrier material such as, but not limited to, water.

In other implementations wherein the photoactivatable fibers have the photoactivatable agent on their surface (i.e., the surface of the fibers that is in contact with the surrounding environment of the fiber), such photoactivatable fibers may be prepared by being sprayed with a photoactivatable agent composition comprising one or more photoactivatable agents and a carrier material.

The carrier material may be any liquid or semi liquid material that is compatible with the photoactivatable agent that is any material that does not affect the photoactive properties of the photoactivatable agent, such as, for example, water. In some other specific examples, the photoactivatable agent composition has a consistency that allows the photoactivatable agent composition to be sprayed onto the fibers.

In the implementations wherein the photoactivatable fibers have the photoactivatable agent incorporated into the fibers, the photoactivatable fibers are prepared by incorporating the photoactivatable agent into the fiber composition. In some examples, the photoactivatable fibers are prepared by extrusion. In some specific implementations, the photoactivatable fibers are prepared by a process which uses spinning. The spinning may be wet, dry, dry jet-wet, melt, gel, or electrospinning. The polymer being spun may be converted into a fluid state. If the polymer is a thermoplastic then it may be melted, otherwise it may be dissolved in a solvent or may be chemically treated to form soluble or thermoplastic derivatives. The molten polymer is then forced through the spinneret, and then it cools to a rubbery state, and then a solidified state. If a polymer solution is used, then the solvent is removed after being forced through the spinneret. A composition of the photoactivatable agent may be added to the polymer in the fluid state or to the melted polymer or to the polymer dissolved into a solvent. Melt spinning may be used for polymers that can be melted. The polymer having the photoactivatable agents dispersed therein solidifies by cooling after being extruded from the spinneret.

The photoactivatable agent may be uniformly or a non-uniformly distributed within the photoactivatable fibers. When the photoactivatable ingredient is uniformly distributed in the photoactivatable fibers, the concentration of photoactivatable agent in the photoactivatable fibers is steady as the photoactivatable fibers disintegrate, whereas when the photoactivatable agent is not uniformly distributed within the photoactivatable fibers, the concentration of the photoactivatable agent in the photoactivatable fibers varies as the photoactivatable fibers disintegrate.

The concentration of the photoactivatable agent to be used may be selected based on the desired intensity and duration of the photoactivity to be emitted from the photoactivatable fibers, and on the desired phototherapeutic, medical or cosmetic effect. For example, some dyes such as xanthene dyes reach a ‘saturation concentration’ after which further increases in concentration do not provide substantially higher emitted fluorescence. Further increasing the photoactivatable agent concentration above the saturation concentration can reduce the amount of activating light passing through the photoactivatable fibers. Therefore, if more fluorescence is required for a certain application than activating light, a high concentration of photoactivatable agent can be used. However, if a balance is required between the emitted fluorescence and the activating light, a concentration close to or lower than the saturation concentration can be chosen.

Suitable photoactivatable agents that may be used in the photoactivatable fibers of the present disclosure include, but are not limited to the following:

Chlorophyll Dyes—

chlorophyll dyes include but are not limited to chlorophyll a; chlorophyll b; chlorophyllin; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Xanthene Derivatives—

xanthene dyes include but are not limited to eosin, eosin Y (2′,4′,5′,7′-tetrabromo-fluorescein, dianion); eosin B (4′,5′-dibromo,2′,7′-dinitr-o-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines that include, but are not limited to, 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Methylene Blue Dyes—

methylene blue derivatives include, but are not limited to, 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-a-mino-phenothiazine; and 1,9-dimethyl-3-diethylamino-7-dibutyl-amino-phenothiazine.

Azo Dyes—

azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more photoactivatable agents of the photoactivatable fibers disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin Green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta O, Magenta I, Magenta II, Magenta DI, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural green 3(chlorophyllin), Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, phycobilins, Phycocyanins, Phycoerythrins. Phycoerythrincyanin (PEC), Phthalocyanines, Pyronin G, Pyronin Y, Quinine, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan DI, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Vitamin B, Water blue I, Water soluble eosin, Xylidine ponceau, or Yellowish eosin.

In certain embodiments, the photoactivatable fibers of the present disclosure may include any of the photoactivatable agents listed above, or a combination thereof, so as to provide a synergistic biophotonic effect. For example, the following synergistic combinations of photoactivatable agents may be used: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine; Eosin Y, Fluorescein and Rose Bengal.

In some examples, the photoactivatable agent is present in the photoactivatable agent composition at a concentration of about 100 g/L, about 50 g/L, about 10 g/L, about 5 g/L, about 1 g/L, about 0.1 g/L, about 0.01 g/L, or about 0.001 g/L of the total volume. Preferably, the photoactivatable agent is present in the photoactivatable agent composition at a concentration of between about 10 g/L and about 100 g/L. In some instances, the photoactivatable agent is present in the photoactivatable agent composition at a concentration that is lower than 0.1 g/L, for example, the photoactivatable agent is present in the photoactivatable agent composition at a concentration in the milligram/L or in the microgram/L range.

In some implementations, there is less than about 15% leaching of the photoactivatable agent out of the photoactivatable fibers of the present disclosure and out of the absorbent biophotonic device of the present disclosure, more preferably less than 10%, more preferably less than 5%, more preferably less than 4%, more preferably less than 3%, more preferably less than 2%, more preferably less than 1%, or even more preferably substantially no leaching of the photoactivatable agent out of the photoactivatable fibers. Leaching of the photoactivatable agent out of the photoactivatable fibers of the present disclosure may be assessed by placing 0.1 g of the photoactivatable fibers in 10 ml of water for a period ranging from about 1 minute to about 3 days and by then measuring the amount of photoactivatable agent in the water.

In some embodiments, the central fibrous layer is composed of a plurality of fibers such as defined herein.

In some implementations, the photoactivatable fibers as defined herein may be woven into a fabric material resulting in a photoactivatable fabric comprising a plurality of photoactivatable fibers. In some implementations, the photoactivatable fabric comprising the photoactivatable fibers exhibits substantially no leaching of the photoactivatable agent.

iii) Bioabsorbent System and Methods of Use

According to one embodiment, the present disclosure relates to an absorbent biophotonic system that may be used for treatment of wounds and/or for healing of wounds. In some implementations, the treatment or the healing of wounds encompasses closure of the wounds. In other implementations, the treatment or healing of wounds encompasses prevention or decrease of infection, contamination or colonization of wounds. In some implementations, the absorbent biophotonic system of the present disclosure emits fluorescence when activated by light. In the same or additional implementations, the absorbent biophotonic system of the present disclosure absorbs fluids and moisture from the wound.

In some implementations of this embodiment, the absorbent biophotonic system comprises an absorbent biophotonic device of the present disclosure together with a light source.

In some implementations of this embodiment, the absorbent biophotonic system comprises an absorbent biophotonic device of the present disclosure and any elements or components to perform a negative-pressure wound therapy (NPWT). In some additional implementations of this embodiment, the absorbent biophotonic system comprises an absorbent biophotonic device of the present disclosure in combination with any elements or components to perform a negative-pressure wound therapy (NPWT) as well as a lighting system (e.g., light source).

As used herein, the expression negative pressure wound therapy (NPWT) refers to a therapeutic technique that uses a vacuum dressing to promote healing of wounds or burns. The therapy involves the controlled application of sub-atmospheric pressure to the local wound environment, using a sealed wound dressing connected to a vacuum pump. General technique for practicing NPWT includes first filing the wound with an absorbent material. The wound is then covered with a fluid-impermeable film (e.g., an occlusive dressing), typically made of, for example, polyurethane. This fluid-impermeable film is preferably clear in color, thin and creates an airtight seal around the wound. A pump is attached to the occlusive dressing and once negative pressure is applied, a vacuum environment is created. The pump can be programmed for strength of suction, amount of time it is to be applied and if it is to be intermittent or continuous. A chamber on the pump collects drainage and moisture is drawn away from the wound site.

In wounds that are not deep enough to accommodate the absorbent material, sterile open weave gauze or other honeycomb dressing textiles can be applied beneath the clear polyurethane film instead. Negative pressure applied can range between about −300 mmHg and about −25 mmHg; or between about −200 mmHg and about −50 mmHg depending upon the wound.

In some embodiments, the absorbent biophotonic system of the present disclosure comprises an absorbent biophotonic device as defined herein and a fluid-impermeable film. In some instances, the absorbent biophotonic device and the fluid-impermeable film are used in combination with a vacuum system and a lighting system.

FIG. 3 shows an absorbent biophotonic system according to one embodiment of the present disclosure. The absorbent biophotonic system (100) comprises an absorbent biophotonic device (120) placed on a wound of a subject's forearm, wherein a bottom layer of absorbent liner (130B) is in direct contact with the wound. The absorbent biophotonic system (100) further comprises fluid-impermeable film (150) covering the absorbent biophotonic device (110). The fluid-impermeable film (150) is of a shape and size sufficient to cover the entirety of the absorbent biophonic device (110) and to cover the skin of the periwound (112). An air-tight seal is created between the fluid-impermeable film (150) and the skin of the periwound (112) to create an airtight seal around the wound.

In some implementations, the fluid-impermeable film (150) is substantially transparent. Is some instances, the fluid-impermeable film (150) is made from transparent flexible plastics such as, but not limited to, polyurethane. Other suitable materials include without limitation synthetic polymeric materials that do not absorb aqueous fluids, including polyolefins, such as polyethylene and polypropylene, polysiloxanes, polyamides, polyesters, and other copolymers and mixtures thereof. The materials used in the fluid-impermeable film (150) may be hydrophobic or hydrophilic. Examples of suitable materials include Transeal® available from DeRoyal and OpSite® available from Smith & Nephew. In order to aid patient comfort and avoid skin maceration, the fluid-impermeable film (150) in certain embodiments is at least partly breathable, such that water vapor is able to pass through without remaining trapped under the dressing.

In some instances, the fluid-impermeable film (150) further comprises an opening (152) for connecting the interior of the fluid-impermeable film (150) to a vacuum system (160) via a vacuum tube (162). In some instances, a drainage tube (not shown) connects the absorbent biophotonic device (110) through an opening in the fluid-impermeable film (150) to a waste container in order to drain excess fluids from the wound and enhance circulation.

In some other instances, the absorbent biophotonic system (110) comprises an illumination tube (172) connecting the interior of the fluid-impermeable film (150) to a light source (170). The illumination tube (172) directs light emitted by the light source (170) in the interior of the absorbent biophotonic device (110) in order to activate the phiotoactivatable agents located in the absorbent biophotonic device (110).

In these embodiments, the combined effect of photoactivation of the light-accepting molecule in the absorbent biophotonic device (110) by the light emitted by the light source (170) as well as of the negative pressure environment created on and around the wound by the vacuum system (160) may provide beneficial wound treating and/or healing conditions.

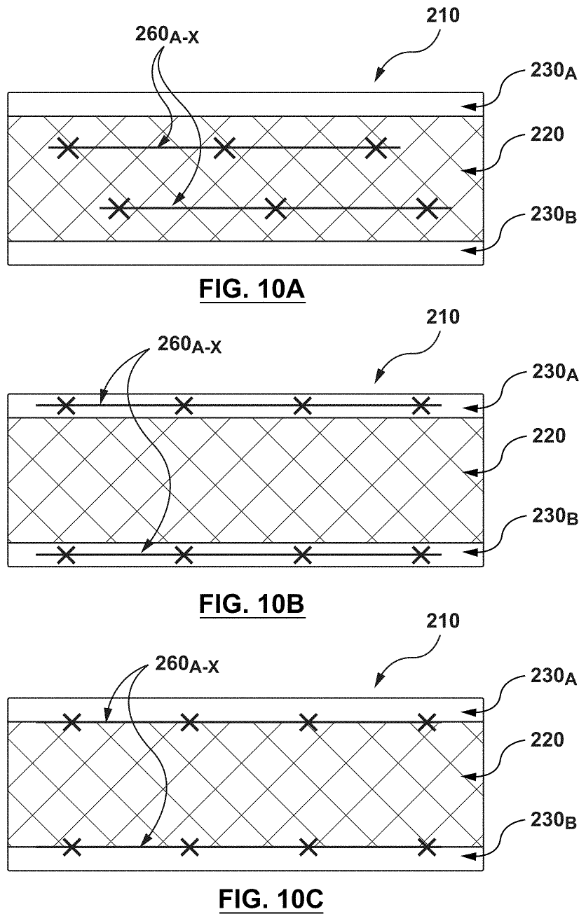

In other embodiments, the light source may be located in proximity of the wound. In such embodiments, such as illustrated in FIGS. 10A, 10B and 10C, the light source is located in the absorbent biophotonoic device (210) itself and may take the form of a plurality of light sources (260a-x) (e.g., LEDs) disposed throughout the photoactivatable core (220) and/or throughout the absorbent liner (230). The plurality of light sources (260a-x) is connected to a power source (not shown) that may be exterior to the absorbent biophotonic device (210).

Any type of source of actinic light can be used with the absorbent biophotonic devices and systems of the present disclosure. For example, any type of halogen, LED or plasma arc lamp, or laser may be suitable. The primary characteristic of suitable sources of actinic light will be that they emit light in a wavelength (or wavelengths) appropriate for activating the light-accepting molecules present in the absorbent biophotonic device. In one embodiment, a LED lamp such as a photocuring device is the source of the actinic light. In another embodiment, the source of the actinic light is a source of light having a wavelength between about 200 nm to 800 nm. In another embodiment, the source of the actinic light is a source of visible light having a wavelength between about 400 nm and 600 nm. In another embodiment, the source of the actinic light is a source of visible light having a wavelength between about 400 nm and 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm². Suitable power density for laser light sources are in the range from about 0.5 mW/cm² to about 0.8 mW/cm².

In some implementations, the light has an energy at the wound surface of between about 0.1 mW/cm² and about 500 mW/cm², or between about 0.1 mW/cm² and about 300 mW/cm², or between about 0.1 mW/cm² and about 200 mW/cm², wherein the energy applied depends at least on the wound being treated, the wavelength of the light, the distance of the wound from the light source and the thickness of the absorbent biophotonic device. In certain embodiments, the light at the wound is between about 1 mW/cm² and about 40 mW/cm², or between about 20 mW/cm² and about 60 mW/cm², or between about 40 mW/cm² and about 80 mW/cm², or between about 60 mW/cm² and about 100 mW/cm², or between about 80 mW/cm² and about 120 mW/cm², or between about 100 mW/cm² and about 140 mW/cm², or between about 30 mW/cm² and about 180 mW/cm², or between about 120 mW/cm² and about 160 mW/cm², or between about 140 mW/cm² and about 180 mW/cm², or between about 160 mW/cm² and about 200 mW/cm², or between about 110 mW/cm² and about 240 mW/cm², or between about 110 mW/cm² and about 150 mW/cm², or between about 190 mW/cm² and about 40 mW/cm².

The activation of the light-accepting molecules may take place almost immediately on illumination (femto- or pico-seconds). A prolonged exposure period may be beneficial to exploit the synergistic effects of the absorbed, reflected and reemitted light of the photoactivatable absorbent fibers of the present disclosure.

In one embodiment, the time of exposure of photoactivatable absorbent fibers to actinic light is a period between about 0.01 minutes and about 90 minutes. In another embodiment, the time of exposure of the photoactivatable absorbent fibers to actinic light is a period between about 1 minute and about 5 minutes. In some other embodiments, the photoactivatable absorbent fibers are illuminated for a period between about 1 minute and about 3 minutes. In certain embodiments, light is applied for a period of about 1 second and about 30 seconds, between about 15 seconds and about 45 seconds, between about 30 seconds and about 60 seconds, between about 0.75 minute and about 1.5 minutes, between about 1 minute and about 2 minutes, between about 1.5 minute and about 2.5 minutes, between about 2 minutes and about 3 minutes, between about 2.5 minutes and about 3.5 minutes, between about 3 minutes and about 4 minutes, between about 3.5 minutes and about 4.5 minutes, between about 4 minutes and about 5 minutes, between about 5 minutes and about 10 minutes, between about 10 minutes and about 15 minutes, between about 15 minutes and about 20 minutes, or between about 20 minutes and about 30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be between about 4 J/cm² and about 60 J/cm², between about 4 J/cm² and about 90 J/cm², between about 10 J/cm² and about 90 J/cm², between about 10 J/cm² and about 60 J/cm², between about 10 J/cm² and about 50 J/cm², between about 10 J/cm² and about 40 J/cm², between about 10 J/cm² and about 30 J/cm², between about 20 J/cm² and about 40 J/cm², between about 15 J/cm² J/cm² and 25 J/cm², or between about 10 J/cm² and about 20 J/cm².

In certain embodiments, the photoactivatable absorbent fibers may be re-illuminated at certain intervals. In yet another embodiment, the source of actinic light is in continuous motion over the treated area for the appropriate time of exposure. In yet another embodiment, the photoactivatable absorbent fibers may be illuminated until the photoactivatable absorbent fibers is at least partially photobleached or fully photobleached.

In certain embodiments, the light-accepting molecules in the photoactivatable absorbent fibers can be photoexcited by ambient light including from the sun and overhead lighting. In certain embodiments, the light-accepting molecules can be photoactivated by light in the visible range of the electromagnetic spectrum. The light can be emitted by any light source such as sunlight, light bulb, an LED device.

In some embodiments, instead of being external to the absorbent biophonic device, the light source may be placed into the absorbent biophotonic device. In such embodiments, the light source may be a plurality of small LED lights that are disposed onto or throughout the photoactivatable core or onto or throughout the absorbent material. In some instances, the light sources may be placed between the fluid-impermeable film and the absorbent material.

In some embodiments, an optical dispersion lens (not shown) may be placed on top of or underneath the fluid-impermeable film at the outlet to direct light towards the treatment site. The optical dispersion lens may allow to direct light toward the area of treatment (e.g., wound).

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more photoactivatable agent present in the composition. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 to 800 nm. In another embodiment, the source of the actinic light is a source of visible light having a wavelength between about 400 and 600 nm. In another embodiment, the source of the actinic light is a source of visible light having a wavelength between about 400 and 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm². Suitable power densities for laser light sources are in the range from about 0.5 mW/cm² to about 0.8 mW/cm².

In some implementations, the light has an energy at the subject's skin surface of between about 0.1 mW/cm² and about 500 mW/cm², or between about 0.1 mW/cm² and about 300 mW/cm², or between about 0.1 mW/cm² and about 200 mW/cm², wherein the energy applied depends at least on the condition being treated, the wavelength of the light, the distance of the skin from the light source and the thickness of the photoactivatable fibers or fabrics. In certain embodiments, the light at the subject's skin is between about 1 mW/cm² and about 40 mW/cm², or between about 20 mW/cm² and about 60 mW/cm², or between about 40 mW/cm² and about 80 mW/cm², or between about 60 mW/cm² and about 100 mW/cm², or between about 80 mW/cm² and about 120 mW/cm², or between about 100 mW/cm² and about 140 mW/cm², or between about 30 mW/cm² and about 180 mW/cm², or between about 120 mW/cm² and about 160 mW/cm², or between about 140 mW/cm² and about 180 mW/cm², or between about 160 mW/cm² and about 200 mW/cm², or between about 110 mW/cm² and about 240 mW/cm², or between about 110 mW/cm² and about 150 mW/cm², or between about 190 mW/cm² and about 240 mW/cm².

The activation of the photoactivatable agents may take place almost immediately on illumination (femto- or pico seconds). A prolonged exposure period may be beneficial to exploit the synergistic effects of the absorbed, reflected and reemitted light of the photoactivatable fibers and fabrics of the present disclosure and its interaction with the tissue being treated. In one embodiment, the time of exposure of photoactivatable fibers or fabrics to actinic light is a period between about 0.01 minutes and about 90 minutes. In another embodiment, the time of exposure of the photoactivatable fibers or fabrics to actinic light is a period between 1 minute and 5 minutes. In some other embodiments, the photoactivatable fibers or fabrics are illuminated for a period between 1 minute and 3 minutes. In certain embodiments, light is applied for a period of between about 1 second and about 30 seconds between about 15 seconds and about 45 seconds, between about 30 seconds and about 60 seconds, between about 0.75 minute and about 1.5 minutes, between about 1 minute and about 2 minutes, between about 1.5 minute and about 2.5 minutes, between about 2 minutes and about 3 minutes, between about 2.5 minutes and about 3.5 minutes, between about 3 minutes and about 4 minutes, between about 3.5 minutes and about 4.5 minutes, between about 4 minutes and about 5 minutes, between about 5 minutes and about 10 minutes, between about 10 minutes and about 15 minutes, between about 15 minutes and about 20 minutes, or between about 20 minutes and about 30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be between about 4 J/cm² and about 60 J/cm², between 4 J/cm² and about 90 J/cm², between 10 J/cm² and about 90 J/cm², between about 10 J/cm² and about 60 J/cm², between about 10 J/cm² and about 50 J/cm², between about 10 J/cm² and about 40 J/cm², between about 10 J/cm² and about 30 J/cm², between about 20 J/cm² and about 40 J/cm², between about 15 J/cm² and about 25 J/cm², or between about 10 J/cm² and about 20 J/cm².

In certain embodiments, the photoactivatable fibers and photoactivatable fabric may be re-illuminated at certain intervals. In yet another embodiment, the source of actinic light is in continuous motion over the treated area for the appropriate time of exposure. In yet another embodiment, the photoactivatable fibers or photoactivatable fabric may be illuminated until the photoactivatable fibers or photoactivatable fabric is at least partially photobleached or fully photobleached.

In certain embodiments, the photoactivatable agents in the photoactivatable fibers or fabrics can be photoexcited by ambient light including from the sun and overhead lighting. In certain embodiments, the photoactivatable agents can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In some instances, a generic NPWT or VAC system and build an add-on parallel unit that will be able to illuminate the photoactivatable based foam without human intervention while the NPWT or VAC system has been initiated.

In some embodiments, the layers of absorbent material may comprise additional agents such as moisturizing agents, antimicrobial agents, urea peroxide or the like that may help in treatment or healing of the wound. It will be appreciated that the layer of absorbent material that is to be in direct contact with the wound should not comprise additional agents that would be toxic to the subject having the wound.

In some embodiments, the absorbent biophotonic material comprising photoactivatable agents do not photoactivatable agents, or light-absorbing molecules, that do not leach over a 24 hour period.

Identification of equivalent devices, systems and methods are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present technology. They are not intended to limit or define the entire scope of this technology. It should be appreciated that the technology is not limited to the particular embodiments described and illustrated herein but includes all modifications and variations falling within the scope of the disclosure as defined in the appended embodiments.

### Example 1: Properties of Absorbent Biophotonic Devices

Experiments were performed to assess several properties of the absorbent biophotonic devices of the present technology. The properties assessed in the experiments were: fluorescence, water absorption capability and leaching of photoactivatable agents out the absorbent biophotonic device. Absorbent biophotonic devices with different configuration, composition and thickness were tested. The absorbent biophotonic devices tested had a photoactivatable core comprising nylon comprising Eosin Y as photoactivatable agent and an absorbent material made of polyethylene. The absorbent biophotonic devices tested were as follows:

Pad #1: 0.80-inch thick foam—one side foam, one side nonwoven

Pad #2: ⅛-inch thick foam—one side foam, one side nonwoven

Pad #3: ¼-inch thick foam—one side foam, one side nonwoven

Pad #4: 1/16-inch foam on each side of nonwoven

Pad #5: ⅛-inch foam on each side of nonwoven

Pad #6: 0.80-inch foam on each side of nonwoven

Pad #7: ¼-inch foam on each side of nonwoven

Pad #8: Doped fiber 116° C. in an oven for 24 hr

Pad #9: Carded and needled blank nylon

Pad #10: Carded and needled doped nylon

Pad #11: Carded and needled blank one side doped on other side

Pad #12: Thermally bonded doped Nylon (200° C. at 20 Psi with speed at 6.2 Hz)

Pad #13: Thermally bonded doped Nylon (200° C. at 10 Psi with speed at 8 Hz)

Leaching of Chromophore—In Vitro Release Test (IVR Test). The release system consisted of a 3-cm diameter compartment with a polycarbonate membrane with pore sizes of 3 μm at the bottom. Inside this compartment, the mesh was placed at the bottom. The foam was cut in circle so that it fits in the 3-cm diameter compartment. This compartment was placed inside of a well with 11 mL of phosphate buffer saline (PBS) such that the membrane just touched the surface of the solution. The sample was illuminated under the Cat-II lamp (blue-green lamp) for 10 minutes and the amount of Eosin Y leached into the PBS solution was determined using the Cary Eclipse. Using Eosin Y in PBS, the fluorescence intensity of each standard solution was plotted against its concentration, making a standard curve. The fluorescence intensity of the leached Eosin Y was measured against the standard curve to determine its concentration. The results are presented in Table 1.

TABLE 1
Amount of Eosin Y Leaching from Foam after
5 min illumination with blue light

| | Fluorescence | Concentration | | |
|---|---|---|---|---|
| Samples | Intensity | PPB | | Average |
| 4 sheets with 0.80″ thick foam-one | 0.1 | BDL* | BDL* | BDL* |
| side foam, one side nonwoven | | | | |
| 4 sheets with ⅛-inch thick foam- | 0.11 | BDL* | BDL* | BDL* |
| one side foam, one side nonwoven | | | | |
| 5 sheets with ¼-inch thick foam- | 0.1 | BDL* | BDL* | BDL* |
| one side foam, one side nonwoven | | | | |
| 2 sheets with 1/16-inch foam | 0.14 | BDL* | BDL* | BDL* |
| on each side of nonwoven | | | | |
| 2 sheets with ⅛-inch foam | 0.25 | BDL* | BDL* | BDL* |
| on each side of nonwoven | | | | |
| 1 sheet with 0.80″ foam | 0.15 | BDL* | BDL* | BDL* |
| on each side of nonwoven | | | | |
| 4 sheets with ¼-inch foam | 0.32 | BDL* | BDL* | BDL* |
| on each side of nonwoven | | | | |
| Dried fiber test; doped fiber | 1.18 | BDL* | BDL* | BDL* |
| 116° C. in an oven for 24 hr. | | | | |
| 3 sheets of carded and | 0.01 | BDL* | BDL* | BDL* |
| needled blank nylon | | | | |
| 3 sheets of carded and | 0.1 | BDL* | BDL* | BDL* |
| needled doped nylon | | | | |
| 1 sheet of carded and needled blank | 0.12 | BDL* | BDL* | BDL* |
| one side doped on other side | | | | |
| 200° C. at 20 psi with speed | 0.57 | BDL* | BDL* | BDL* |
| at 6.2 Hz | | | | |
| 200° C. at 10 psi with speed | 6.92 | BDL* | 6.88 | 5.94** |
| set to 8.0 Hz | | | | |

*BDL = Below Detection Limit (5 ppb)
**Average = BDL/2 + 6.88/2 = 5.94 ppb
The in vitro release test shows that there is no leaching coming from the foam.

Lamp and Fluorescence Measurements—

To measure the fluorescence, a 3×3 cm square of foam was cut and placed 5 cm away from the blue Terra lamp. The sample was illuminated for a total of 7.5 minutes, with measurements taken every 30 seconds. Below the sample, a SP-100 spectroradiometer along with a filter measures the power density spectra. The results are presented in Table 2.

TABLE 2
Fluorescence of Foam after 5 min illumination
with Blue Terra Lamp.

| | Fluorescence |
|---|---|
| Sample | (J/cm²) |
| 4 sheets with 0.080-inch thick foam-one | 0.98 |
| side foam, one side nonwoven | |
| 4 sheets with ⅛-inch thick foam-one | 0.83 |
| side foam, one side nonwoven | |
| 5 sheets with ¼-inch thick foam-one | 0.81 |
| side foam, one side nonwoven | |
| 2 sheets with 1/16-inch foam on | 1.43 |
| each side of nonwoven | |
| 2 sheets with ⅛-inch foam on | 0.97 |
| each side of nonwoven | |
| 1 sheet with 0.080-inch foam on | 1.11 |
| each side of nonwoven | |
| 4 sheets with ¼-inch foam on | 0.64 |
| each side of nonwoven | |
| Dried fiber test; doped fiber 116° C. | 0.77 |
| in an oven for 24 hr | |
| 3 sheets of carded and needled blank nylon | 0.01 |
| 3 sheets of carded and needled doped nylon | 1.02 |
| 1 sheet of carded and needled blank | 0.52 |
| one side doped on other side | |
| 200° C. at 20 psi with speed at 6.2 Hz | 1.75 |
| 200° C. at 10 psi with speed set to 8.0 Hz | 1.70 |

The optimal fluorescence was obtained with the foams heated at 200° Celsius. It was observed that the fluorescence of the samples decreased with the thickness of the foam, which blocks more light. Furthermore, the samples with foams on each side of nonwoven were more fluorescent than the samples with only a layer of foam on one side. This may be due to the dispersion of light passing by the white foam.

Foam Water Retention—

Water retention test was made on the foam, each sample was cut in a 3×3 cm and immersed in water for 1 minute. The foam weight was taken before and after immersion and the difference was considered as the water retention. Results are shown in Table 3.

TABLE 3
Water absorption by Foam

| Water absorption by Foam for 1 min | | | |
|---|---|---|---|
| | weight | weight with | Absorption |
| Sample | (single)-wi | water-wf | wf-wi(g) |
| FOAM BAG A WHITE | 0.24 | 0.34 | 0.1 |
| FOAM BAG B WHITE | 0.29 | 0.43 | 0.14 |
| FOAM BLACK 100% | 0.32 | 1.43 | 1.11 |
| FOAM BLACK (25%) | 0.12 | 0.34 | 0.22 |
| FOAM BLACK (50%) | 0.21 | 0.64 | 0.43 |
| 1 sheet with 0.80″ foam | 0.22 | 0.29 | 0.06 |
| on each side of nonwoven | | | |
| 2 sheets with ⅛-inch foam | 0.31 | 0.54 | 0.23 |
| on each side of nonwoven | | | |
| 2 sheets with 1/16-inch foam | 0.21 | 0.39 | 0.18 |
| on each side of nonwoven | | | |
| 4 sheets with ¼-inch foam | 0.60 | 0.83 | 0.24 |
| on each side of nonwoven | | | |

The data show that the water retention increases as we increase the thickness of the foam.

In conclusion, all the foam did not leach and the best fluorescent among them was observed with the foam having the lowest thickness. Also, the absorbent biophotonic pads with foams on each side were more fluorescent than the absorbent biophotonic pads with only one layer of foam on one side.

### Example 2: Photoactivity of Absorbent Biophotonic Devices

The photoactivity of absorbent biophotonic devices according to the present technology was assessed. The absorbent biophotonic devices outlined in Table 4 were tested:

TABLE 4
Absorbent biophotonic devices

| Absorbent | |
|---|---|
| biophotonic | |
| device | Composition |
| A | Absorbent material: polyurethane foam, 3 mm thick |
| | Photoactivatable core: Nylon fibers comprising Eosin Y |
| | Actinic light: Blue light |
| B | Absorbent material: polyurethane foam, 9 mm thick |
| | Photoactivatable core: Nylon fibers comprising Eosin Y |
| | Actinic light: Blue light |

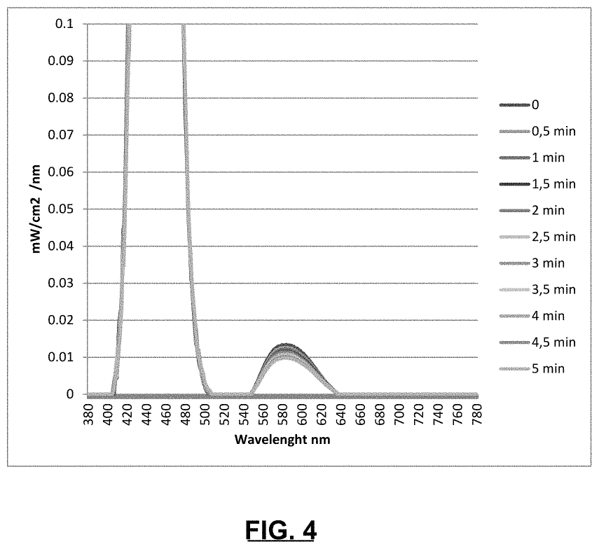

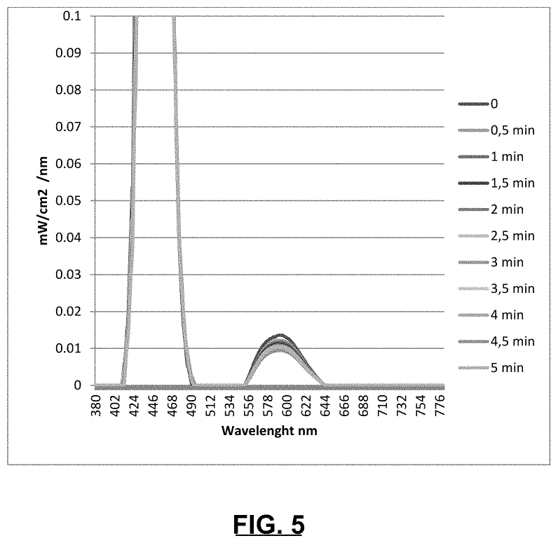

For both absorbent biophonic devices A and B, the photoactivatable core was placed (i.e., sandwiched) between two layers of absorbent material. Absorbent biophonic devices A and B were each exposed to blue light using KLOX Thera® Lamp for a period of 5 minutes. The light source was placed at a distance of 5 cm from the absorbent biophonic devices. Fluorescence emitted by absorbent biophonic devices A and B are indicated in Tables 5 and 6 respectively as well as in FIG. 4 and FIG. 5 respectively.

TABLE 5
Fluorescence emitted from photoactivated absorbent biophotonic device A

| 3 mm Polyurethane Foam | | mW/cm² at 5 cm | | | | | | |
|---|---|---|---|---|---|---|---|---|
| with Eosin-KTL | | 0 | 0.5 min | 1 min | 1.5 min | 2 min | 2.5 min | |
| Lamp | 400-518 | 45.89 | 45.87 | 45.49 | 45.08 | 44.66 | 44.27 | |
| Fluoresc. | 519-760 | 0.73 | 0.69 | 0.66 | 0.64 | 0.63 | 0.60 | |
| total | 400-760 | 46.61 | 46.55 | 46.14 | 45.72 | 45.28 | 44.86 | |
| % fluorescence | | 1.6% | 1.5% | 1.4% | 1.4% | 1.4% | 1.3% | |
| Purple | (400)- | 22.32 | 22.03 | 21.66 | 21.30 | 20.95 | 20.61 | |
| | 450 | | | | | | | |
| Blue | 450-500 | 23.55 | 23.82 | 23.81 | 23.76 | 23.69 | 23.64 | |
| Green | 500-570 | 0.13 | 0.12 | 0.12 | 0.12 | 0.12 | 0.11 | |
| Yellow | 570-591 | 0.27 | 0.25 | 0.24 | 0.23 | 0.23 | 0.22 | |
| Orange | 591-610 | 0.22 | 0.208 | 0.20 | 0.19 | 0.18 | 0.17 | |
| Red | 610-760 | 0.11 | 0.109 | 0.10 | 0.10 | 0.09 | 0.09 | |
| total | 400-700 | 46.63 | 46.56 | 46.15 | 45.73 | 45.29 | 44.87 | |
| 3 mm Polyurethane Foam | | mW/cm² at 5 cm | | | | | | |
| with Eosin-KTL | | 3 min | 3.5 min | 4 min | 4.5 min | 5 min | J/cm2 | |
| Lamp | 400-518 | 43.84 | 43.39 | 43.08 | 42.72 | 42.40 | 13.33 | 98.6% |
| Fluoresc. | 519-760 | 0.58 | 0.57 | 0.56 | 0.54 | 0.54 | 0.19 | 1.4% |
| total | 400-760 | 44.41 | 43.95 | 43.63 | 43.26 | 42.94 | 13.51 | 100.0% |
| % fluorescence | | 1.3% | 1.3% | 1.3% | 1.3% | 1.3% | 0.01 | 1.4% |
| Purple | (400)- | 20.26 | 19.91 | 19.64 | 19.37 | 19.11 | 6.24 | 46.2% |
| | 450 | | | | | | | |
| Blue | 450-500 | 23.56 | 23.45 | 23.41 | 23.32 | 23.27 | 7.08 | 52.4% |
| Green | 500-570 | 0.10 | 0.11 | 0.11 | 0.10 | 0.10 | 0.04 | 0.3% |
| Yellow | 570-591 | 0.21 | 0.21 | 0.20 | 0.20 | 0.19 | 0.07 | 0.5% |
| Orange | 591-610 | 0.17 | 0.17 | 0.16 | 0.16 | 0.16 | 0.06 | 0.4% |
| Red | 610-760 | 0.09 | 0.08 | 0.08 | 0.08 | 0.08 | 0.03 | 0.2% |
| total | 400-700 | 44.43 | 43.96 | 43.64 | 43.27 | 42.95 | 13.52 | 100.0% |

TABLE 6
Fluorescence emitted from photoactivated absorbent biophotonic device A

| 9 mm Polyurethane Foam | | mW/cm² at 5 cm | | | | | | |
|---|---|---|---|---|---|---|---|---|
| with Eosin-KTL | | 0 | 0.5 min | 1 min | 1.5 min | 2 min | 2.5 min | |
| Lamp | 400-518 | 20.73 | 20.64 | 20.33 | 20.04 | 19.75 | 19.45 | |
| Fluoresc. | 519-760 | 0.75 | 0.68 | 0.65 | 0.63 | 0.60 | 0.59 | |
| total | 400-760 | 21.48 | 21.32 | 20.98 | 20.66 | 20.35 | 20.03 | |
| % fluores- | | 3.5% | 3.2% | 3.1% | 3.0% | 3.0% | 2.9% | |
| cence | | | | | | | | |
| Purple | (400)- | 10.74 | 10.50 | 10.25 | 9.99 | 9.76 | 9.54 | |
| | 450 | | | | | | | |
| Blue | 450-500 | 9.98 | 10.13 | 10.07 | 10.03 | 9.97 | 9.90 | |
| Green | 500-570 | 0.08 | 0.07 | 0.07 | 0.06 | 0.06 | 0.06 | |
| Yellow | 570-591 | 0.25 | 0.22 | 0.21 | 0.21 | 0.20 | 0.19 | |
| Orange | 591-610 | 0.25 | 0.22 | 0.21 | 0.21 | 0.20 | 0.19 | |
| Red | 610-760 | 0.16 | 0.15 | 0.15 | 0.14 | 0.14 | 0.13 | |
| total | 400-700 | 21.49 | 21.33 | 20.99 | 20.67 | 20.36 | 20.05 | |
| 9 mm Polyurethane Foam | | mW/cm² at 5 cm | | | | | | |
| with Eosin-KTL | | 3 min | 3.5 min | 4 min | 4.5 min | 5 min | J/cm2 | |
| Lamp | 400-518 | 19.15 | 18.90 | 18.65 | 18.42 | 17.76 | 5.88 | 96.9% |
| Fluoresc. | 519-760 | 0.57 | 0.55 | 0.55 | 0.53 | 0.57 | 0.18 | 3.0% |
| total | 400-760 | 19.71 | 19.44 | 19.20 | 18.94 | 18.32 | 6.06 | 100.0% |
| % fluores- | | 2.9% | 2.8% | 2.9% | 2.8% | 3.1% | 0.03 | 3.0% |
| cence | | | | | | | | |
| Purple | (400)- | 9.31 | 9.11 | 8.93 | 8.75 | 8.48 | 2.91 | 47.9% |
| | 450 | | | | | | | |
| Blue | 450-500 | 9.84 | 9.78 | 9.71 | 9.66 | 9.27 | 2.97 | 49.0% |
| Green | 500-570 | 0.06 | 0.05 | 0.05 | 0.05 | 0.06 | 0.02 | 0.3% |
| Yellow | 570-591 | 0.19 | 0.18 | 0.18 | 0.17 | 0.19 | 0.06 | 1.0% |
| Orange | 591-610 | 0.18 | 0.18 | 0.18 | 0.17 | 0.19 | 0.06 | 1.0% |
| Red | 610-760 | 0.13 | 0.12 | 0.13 | 0.12 | 0.13 | 0.04 | 0.7% |
| total | 400-700 | 19.73 | 19.45 | 19.21 | 18.95 | 18.34 | 6.07 | 100.0% |

### Example 2: Photoactivity of Absorbent Biophotonic Systems

Experiments were performed to the photoactivity of an absorbent biophotonic system according embodiments of the present technology wherein the absorbent biophotonic devices of the present technology were used in combination with a negative pressure wound treatment system.

The absorbent biophotonic system was tested for the efficiency of its photoactivating agent at a negative Pressure of −50 mmHg, −100 mmHg or −200 mmHg. A 1 cm thick photoactivatable core was prepared using nylon fibers comprising Eosin Y as light-accepting molecule. A top polyurethane foam absorbent liner having a thickness of ⅛ of an inch was placed on the top surface of the photoactivatable core and a bottom polyurethane foam absorbent liner also having a thickness of ⅛ of an inch was placed on the top surface of the photoactivatable core. Table 7 below indicates the composition of the absorbent biophotonic devices that were used in the experiments as well as the parameters of the vacuum system.

TABLE 7
Composition of absorbent biophotonic systems

| Absorbent | | | |
|---|---|---|---|
| Biophotonic | Biophotonic Pad | | Vacuum- |
| System | Photoactivatable core | Absorbent material | System |
| 1 | Nylon fibers with Eosin Y | Polyurethane; thickness of ⅛ of an | −50 mmHg |
| | (1 cm thick) | inch (2X for top and bottom) | |
| 2 | Nylon fibers with Eosin Y | Polyurethane; thickness of ⅛ of an | −100 mmHg |
| | (1 cm thick) | inch (2X for top and bottom) | |
| 3 | Nylon fibers with Eosin Y | Polyurethane; thickness of ⅛ of an | −200 mmHg |
| | (1 cm thick) | inch (2X for top and bottom) | |

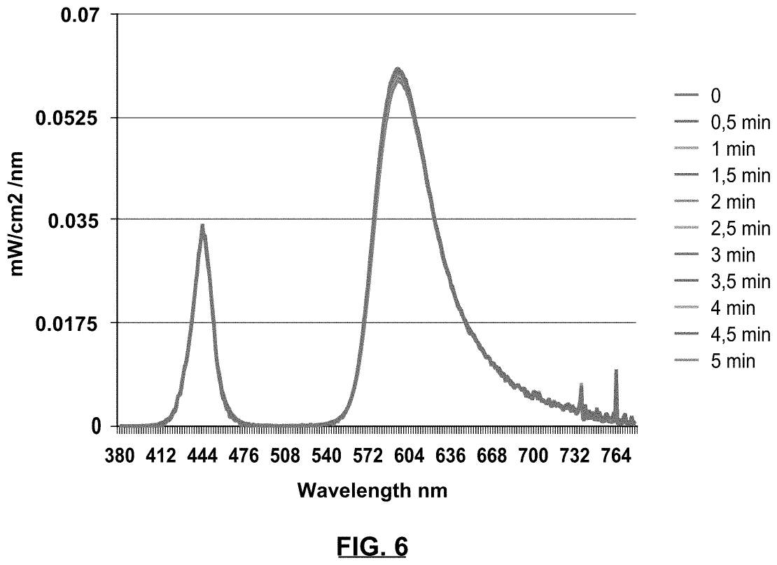

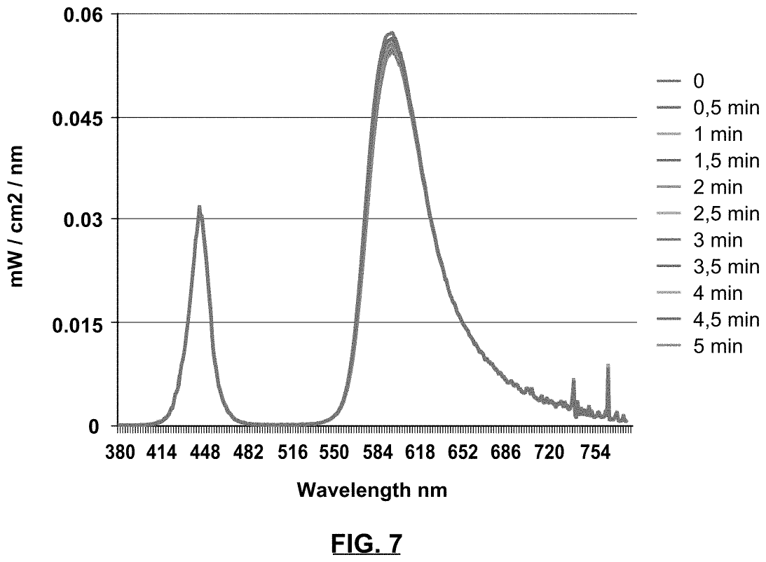

Table 8 as well as FIG. 6 show the composition of the light emitted by the absorbent biophotonic device of absorbent biophotonic system 1. Table 9 as well as FIG. 7 show the composition of the light emitted by the absorbent biophotonic device of absorbent biophotonic system 2. Table 10 as well as FIG. 8 show the composition of the light emitted by the absorbent biophotonic device of absorbent biophotonic system 3.

TABLE 8
Fluorescence emitted from photoactivated absorbent system 1

| rep. 50 mm VAC | | mW/cm² at 0 cm | | | | | |
|---|---|---|---|---|---|---|---|
| 1/8PU 1 cm foam D1 | | 0 | 0.5 min | 1 min | 1.5 min | 2 min | 2.5 min |
| Lamp | 400-518 | 0.66 | 0.70 | 0.70 | 0.70 | 0.70 | 0.70 |
| Fluoresc. | 519-760 | 3.99 | 4.07 | 4.06 | 4.04 | 4.03 | 4.01 |
| total | 400-760 | 4.65 | 4.76 | 4.75 | 4.73 | 4.72 | 4.71 |
| % fluorescence | | 85.8% | 85.3% | 85.3% | 85.3% | 85.2% | 85.1% |
| Purple | (400)-450 | 0.49 | 0.51 | 0.51 | 0.51 | 0.51 | 0.51 |
| Blue | 450-500 | 0.16 | 0.17 | 0.18 | 0.17 | 0.18 | 0.18 |
| Green | 500-570 | 0.13 | 0.13 | 0.13 | 0.13 | 0.13 | 0.13 |
| Yellow | 570-591 | 0.86 | 0.85 | 0.84 | 0.84 | 0.83 | 0.83 |
| Orange | 591-610 | 1.14 | 1.15 | 1.15 | 1.14 | 1.14 | 1.13 |
| Red | 610-760 | 1.90 | 1.96 | 1.96 | 1.96 | 1.96 | 1.95 |
| total | 400-700 | 4.71 | 4.82 | 4.81 | 4.78 | 4.78 | 4.76 |
| rep. 50 mm VAC | | mW/cm² at 0 cm | | | | | |
| 1/8PU 1 cm foam D1 | | 3 min | 3.5 min | 4 min | 4.5 min | 5 min | J/cm2 |
| Lamp | 400-518 | 0.70 | 0.70 | 0.70 | 0.70 | 0.70 | 0.21 |
| Fluoresc. | 519-760 | 4.00 | 3.98 | 3.97 | 3.96 | 3.94 | 1.20 |
| total | 400-760 | 4.69 | 4.67 | 4.66 | 4.65 | 4.63 | 1.41 |
| % fluorescence | | 85.1% | 85.1% | 85.1% | 85.0% | 85.0% | 0.85 |
| Purple | (400)-450 | 0.51 | 0.51 | 0.51 | 0.51 | 0.51 | 0.15 |
| Blue | 450-500 | 0.18 | 0.18 | 0.18 | 0.18 | 0.18 | 0.05 |
| Green | 500-570 | 0.13 | 0.13 | 0.13 | 0.13 | 0.13 | 0.04 |
| Yellow | 570-591 | 0.82 | 0.82 | 0.81 | 0.81 | 0.80 | 0.25 |
| Orange | 591-610 | 1.13 | 1.13 | 1.12 | 1.12 | 1.11 | 0.34 |
| Red | 610-760 | 1.95 | 1.94 | 1.94 | 1.94 | 1.92 | 0.58 |
| total | 400-700 | 4.75 | 4.73 | 4.72 | 4.71 | 4.68 | 1.43 |

TABLE 9
Fluorescence emitted from photoactivated absorbent system 2

| rep. 100 mm VAC | | mW/cm² at 0 cm | | | | | |
|---|---|---|---|---|---|---|---|
| 1/8PU 1 cm foam D1 | | 0 | 0.5 min | 1 min | 1.5 min | 2 min | 2.5 min |
| Lamp | 400-518 | 0.65 | 0.64 | 0.65 | 0.65 | 0.65 | 0.65 |
| Fluoresc. | 519-760 | 3.87 | 3.82 | 3.80 | 3.79 | 3.77 | 3.75 |
| total | 400-760 | 4.51 | 4.46 | 4.45 | 4.44 | 4.41 | 4.40 |
| % fluorescence | | 85.7% | 85.6% | 85.4% | 85.3% | 85.2% | 85.2% |
| Purple | (400)-450 | 0.48 | 0.47 | 0.48 | 0.48 | 0.48 | 0.47 |
| Blue | 450-500 | 0.16 | 0.16 | 0.16 | 0.17 | 0.17 | 0.17 |
| Green | 500-570 | 0.15 | 0.15 | 0.15 | 0.14 | 0.14 | 0.14 |
| Yellow | 570-591 | 0.89 | 0.87 | 0.86 | 0.85 | 0.84 | 0.83 |
| Orange | 591-610 | 1.07 | 1.06 | 1.06 | 1.05 | 1.05 | 1.05 |
| Red | 610-760 | 1.78 | 1.77 | 1.77 | 1.77 | 1.77 | 1.76 |
| total | 400-700 | 4.56 | 4.51 | 4.50 | 4.49 | 4.46 | 4.45 |
| rep. 100 mm VAC | | mW/cm² at 0 cm | | | | | |
| 1/8PU 1 cm foam D1 | | 3 min | 3.5 min | 4 min | 4.5 min | 5 min | J/cm2 |
| Lamp | 400-518 | 0.65 | 0.65 | 0.66 | 0.66 | 0.66 | 0.20 |
| Fluoresc. | 519-760 | 3.74 | 3.72 | 3.71 | 3.70 | 3.69 | 1.13 |
| total | 400-760 | 4.39 | 4.37 | 4.36 | 4.35 | 4.35 | 1.33 |
| % fluorescence | | 85.1% | 85.1% | 85.0% | 84.9% | 84.8% | 0.85 |
| Purple | (400)-450 | 0.48 | 0.48 | 0.48 | 0.48 | 0.48 | 0.14 |
| Blue | 450-500 | 0.17 | 0.17 | 0.17 | 0.17 | 0.17 | 0.05 |
| Green | 500-570 | 0.14 | 0.14 | 0.14 | 0.13 | 0.13 | 0.04 |
| Yellow | 570-591 | 0.82 | 0.82 | 0.81 | 0.80 | 0.80 | 0.25 |
| Orange | 591-610 | 1.04 | 1.04 | 1.04 | 1.03 | 1.03 | 0.32 |
| Red | 610-760 | 1.76 | 1.76 | 1.76 | 1.76 | 1.76 | 0.53 |
| total | 400-700 | 4.44 | 4.42 | 4.41 | 4.40 | 4.40 | 1.34 |

TABLE 10
Fluorescence emitted from photoactivated absorbent system 1

| rep. 200 mmHg VAC | | mW/cm² at 0 cm | | | | | |
|---|---|---|---|---|---|---|---|
| 1/8PU 1 cm foam D1 | | 0 | 0.5 min | 1 min | 1.5 min | 2 min | 2.5 min |
| Lamp | 400-518 | 0.63 | 0.63 | 0.63 | 0.63 | 0.63 | 0.63 |
| Fluoresc. | 519-760 | 3.57 | 3.55 | 3.53 | 3.52 | 3.50 | 3.48 |
| total | 400-760 | 4.20 | 4.17 | 4.16 | 4.14 | 4.12 | 4.11 |
| % fluorescence | | 85.0% | 85.0% | 84.9% | 84.8% | 84.7% | 84.7% |
| Purple | (400)-450 | 0.45 | 0.45 | 0.45 | 0.45 | 0.45 | 0.45 |
| Blue | 450-500 | 0.17 | 0.16 | 0.17 | 0.17 | 0.17 | 0.17 |
| Green | 500-570 | 0.14 | 0.13 | 0.13 | 0.13 | 0.13 | 0.13 |
| Yellow | 570-591 | 0.80 | 0.79 | 0.78 | 0.77 | 0.77 | 0.76 |
| Orange | 591-610 | 0.98 | 0.98 | 0.97 | 0.97 | 0.97 | 0.96 |
| Red | 610-760 | 1.68 | 1.67 | 1.67 | 1.66 | 1.66 | 1.66 |
| total | 400-700 | 4.24 | 4.22 | 4.20 | 4.19 | 4.17 | 4.16 |
| rep. 200 mmHg VAC | | mW/cm² at 0 cm | | | | | |
| 1/8PU 1 cm foam D1 | | 3 min | 3.5 min | 4 min | 4.5 min | 5 min | J/cm2 |
| Lamp | 400-518 | 0.63 | 0.63 | 0.63 | 0.63 | 0.63 | 0.19 |
| Fluoresc. | 519-760 | 3.47 | 3.46 | 3.44 | 3.43 | 3.42 | 1.05 |
| total | 400-760 | 4.09 | 4.08 | 4.06 | 4.06 | 4.04 | 1.24 |
| % fluorescence | | 84.7% | 84.6% | 84.6% | 84.5% | 84.5% | 0.85 |
| Purple | (400)-450 | 0.45 | 0.45 | 0.45 | 0.45 | 0.45 | 0.14 |
| Blue | 450-500 | 0.17 | 0.17 | 0.17 | 0.17 | 0.17 | 0.05 |
| Green | 500-570 | 0.13 | 0.13 | 0.13 | 0.13 | 0.12 | 0.04 |
| Yellow | 570-591 | 0.75 | 0.75 | 0.74 | 0.74 | 0.74 | 0.23 |
| Orange | 591-610 | 0.96 | 0.96 | 0.95 | 0.95 | 0.95 | 0.29 |
| Red | 610-760 | 1.65 | 1.65 | 1.64 | 1.64 | 1.64 | 0.50 |
| total | 400-700 | 4.14 | 4.13 | 4.11 | 4.10 | 4.09 | 1.25 |

### Example 3: Leaching of Photoactivatable Agent Out of Absorbent Biophotonic Devices

An experiment was performed to assess the level of light-accepting molecules leaching out of the absorbent biophotonic device of the present disclosure. Absorbent biophotonic devices with different composition were placed in contact with a liquid for 24 hours. The amount of light-absorbing molecules present in the liquid after 24 hours was measured. The results are presented in Table 11 and Table 12.

TABLE 11
Composition of the absorbent biophotonic pads tested for leaching of light-activating molecules

| Absorbent | | | |
|---|---|---|---|
| Biophotonic | | | |
| Pad | Photoactivatable Core | Absorbent liners | Needled |
| A | 50 g of nylon fibers (sheet core: 90% | top liner + bottom polyurethane | needled/punctured |
| | DOPPED with chromophore + 10% BiCo) | foam liners ( 1/32 inches each) | through both |
| | | | absorbent liners |
| B | 50 g of nylon fibers (sheet core: 90% | top liner + bottom polyurethane | needled/punctured |
| | DOPPED with chromophore + 10% | foam liners ( 1/32 inches each) | through both |
| | BLANK/no chromophore) | | absorbent liners |
| C | 50 g of nylon fibers (sheet core: 90% | top liner + bottom polyurethane | needled/punctured |
| | DOPPED with chromophore + 10% | foam liners (⅛ inches each) | through both |
| | BLANK/no chromophore) | | absorbent liners |
| D | 75 g of nylon fibers (sheet core: 90% | top liner + bottom polyurethane | needled/punctured |
| | DOPPED with chromophore + 10% BiCo) | foam liners (⅛ inches each) | through both |
| | | | absorbent liners |
| E | 75 g of nylon fibers (sheet core: 90% | top liner + bottom polyurethane | needled/punctured |
| | DOPPED with chromophore + 10% | foam liners (⅛ inches each) | through both |
| | BLANK/no chromophore) | | absorbent liners |
| F | 50 g of nylon fibers (100% DOPPED with | top liner + bottom polyurethane | Needled using |
| | chromophore) | foam liners (⅛ inches each) | Absorbent liner 1 |

Chromophore = Eosin Y + Rose Bengal

TABLE 12
Leaching of light-accepting molecules out of the
indicated absorbent biophotonic pads

| Absorbent | | Eosin Y | Rose Bengal |
|---|---|---|---|
| Biophotonic | Fluorescence | leached out | leached out |
| Pad | emitted | after 24 hrs (ppb) | after 24 hrs (ppb) |
| A | 1.37 | 1000 | 150 |
| B | 1.37 | 1200 | 215 |
| C | 0.7 | 0.3 | 22 |
| D | 0.6 | 1.2 | 18 |
| E | 0.6 | 2.5 | 22 |
| F | 0.6 | 1.2 | 25 |

### Example 4: Leaching of Photoactivatable Agent Out of Absorbent Biophotonic Devices

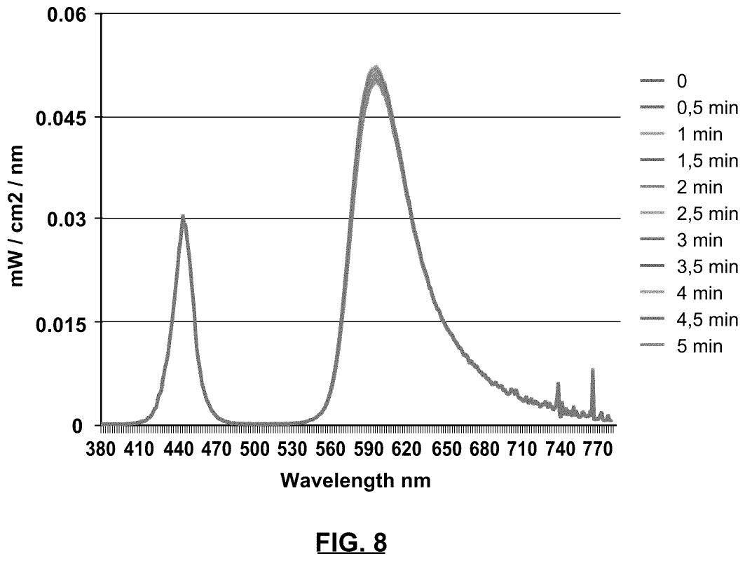

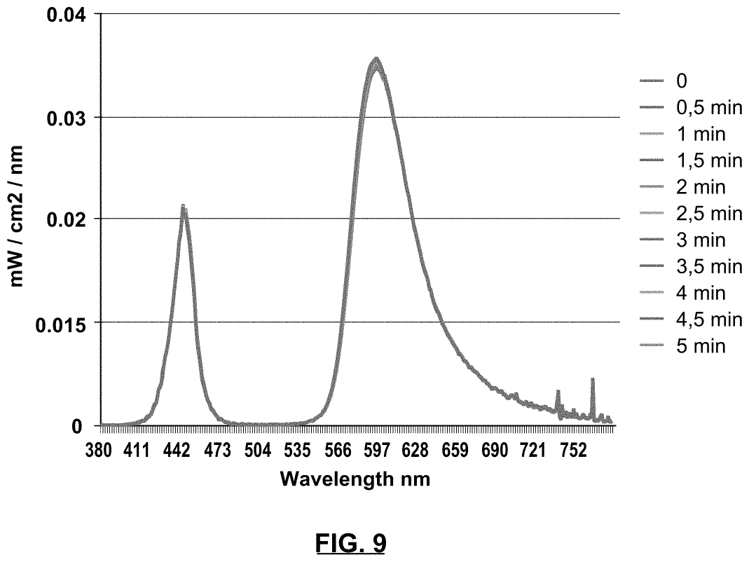

An experiment was performed with absorbent biophotonic devices of the present disclosure to measure leaching of the photoactivating agent from the absorbent biophotonic device. In this experiment, the absorbent biophotonic device comprising 50 grams of photoactivatable was placed in contact with a liquid for 24. The amount of photoactivatable agent present in the liquid after 24 hours was measured. The results are presented in Table 13 and in FIG. 9.

TABLE 13
Leaching of Photoactivatable agent out of absorbent biophotonic devices

| d1 50 g 90% dopped + | | mW/cm² at 5 cm | | | | | |
|---|---|---|---|---|---|---|---|
| 10% blank needled | | 0 | 0.5 min | 1 min | 1.5 min | 2 min | 2.5 min |
| Lamp | 400-518 | 0.46 | 0.46 | 0.47 | 0.47 | 0.47 | 0.47 |
| Fluoresc. | 519-760 | 2.32 | 2.32 | 2.31 | 2.30 | 2.30 | 2.29 |
| total | 400-760 | 2.78 | 2.78 | 2.77 | 2.76 | 2.76 | 2.76 |
| % fluorescence | | 83.5% | 83.4% | 83.2% | 83.1% | 83.1% | 83.0% |
| Purple | (400)-450 | 0.33 | 0.33 | 0.33 | 0.33 | 0.33 | 0.33 |
| Blue | 450-500 | 0.12 | 0.12 | 0.13 | 0.13 | 0.13 | 0.13 |
| Green | 500-570 | 0.08 | 0.08 | 0.08 | 0.08 | 0.08 | 0.08 |
| Yellow | 570-591 | 0.52 | 0.52 | 0.50 | 0.50 | 0.50 | 0.50 |
| Orange | 591-610 | 0.67 | 0.67 | 0.66 | 0.66 | 0.66 | 0.66 |
| Red | 610-760 | 1.06 | 1.07 | 1.07 | 1.07 | 1.07 | 1.07 |
| total | 400-700 | 2.81 | 2.81 | 2.80 | 2.80 | 2.80 | 2.79 |
| d1 50 g 90% dopped + | | mW/cm² at 5 cm | | | | | |
| 10% blank needled | | 3 min | 3.5 min | 4 min | 4.5 min | 5 min | J/cm2 |
| Lamp | 400-518 | 0.47 | 0.47 | 0.47 | 0.47 | 0.47 | 0.14 |
| Fluoresc. | 519-760 | 2.29 | 2.28 | 2.28 | 2.27 | 2.27 | 0.69 |
| total | 400-760 | 2.75 | 2.75 | 2.75 | 2.74 | 2.73 | 0.83 |
| % fluorescence | | 82.9% | 82.9% | 82.9% | 82.8% | 82.8% | 0.83 |
| Purple | (400)-450 | 0.33 | 0.33 | 0.33 | 0.33 | 0.33 | 0.10 |
| Blue | 450-500 | 0.13 | 0.13 | 0.13 | 0.13 | 0.13 | 0.04 |
| Green | 500-570 | 0.08 | 0.08 | 0.08 | 0.07 | 0.07 | 0.02 |
| Yellow | 570-591 | 0.49 | 0.49 | 0.49 | 0.49 | 0.48 | 0.15 |
| Orange | 591-610 | 0.66 | 0.66 | 0.66 | 0.65 | 0.65 | 0.20 |
| Red | 610-760 | 1.07 | 1.07 | 1.07 | 1.07 | 1.06 | 0.32 |
| total | 400-700 | 2.79 | 2.78 | 2.78 | 2.77 | 2.76 | 0.84 |

Any feature of any embodiment discussed herein may be combined with any feature of any other embodiment discussed herein in some examples of implementation.

Certain additional elements that may be needed for operation of certain embodiments have not been described or illustrated as they are assumed to be within the purview of those skilled in the art. Moreover, certain embodiments may be free of, may lack and/or may function without any element that is not specifically disclosed herein.

Although various embodiments and examples have been presented, this was for the purpose of describing, but not limiting, the invention. Various modifications and enhancements will become apparent to those skilled in the art and are within the scope of the invention, which is defined by the appended claims.

All documents referred to herein are incorporated by reference.

1. An absorbent biophotonic device comprising:
an photoactivatable core; and
at least one absorbent liner disposed on at least a part of the photoactivatable core; wherein the photoactivatable core is photoactivated upon exposure to light to emit fluorescence.

2. An absorbent biophotonic device for treatment and/or healing of a wound, the absorbent biophotonic device comprising:
a photoactivatable core; and
at least one absorbent liner disposed on at least a part the photoactivatable core;
wherein the photoactivatable core is photoactivated upon exposure to light to emit fluorescence.

3. The absorbent biophotonic device as defined in claim 1, wherein the photoactivatable core comprises light-accepting molecules.

4. The absorbent biophotonic device as defined in claim 1, wherein the light-accepting molecules are Eosin.

5. The absorbent biophotonic device as defined in claim 4, wherein the Eosin is Eosin Y.

6. The absorbent biophotonic device as defined in claim 4, wherein the Eosin is Eosin Y and Eosin B.

7. The absorbent biophotonic device as defined in claim 1, wherein the light-accepting molecules are Eosin Y and Fluorescein.

8. The absorbent biophotonic device as defined in claim 1, wherein the light-accepting molecules are Eosin Y and Rose Bengal.

9. The absorbent biophotonic device as defined in claim 1, wherein the photoactivatable core comprises nylon.

10. The absorbent biophotonic device as defined in claim 1, wherein the at least one absorbent liner is a bottom absorbent liner.

11. The absorbent biophotonic device as defined in claim 1, wherein the at least one absorbent liner is a top absorbent liner.

12. The absorbent biophotonic device as defined in claim 1, wherein the absorbent biophotonic device comprises two absorbent liners.

13. The absorbent biophotonic device as defined in claim 12, wherein the a photoactivatable core is disposed between the two absorbent liners.

14. The absorbent biophotonic device as defined in claim 1, wherein the at least one absorbent liner is made of foam.

15. The absorbent biophotonic device as defined in claim 14, wherein the foam is a polyurethane foam.

16. The absorbent biophotonic device as defined in claim 15, wherein the polyurethane foam is a reticulated polyurethane foam.

17. The absorbent biophotonic device as defined in claim 14, wherein the foam is a polyethylene foam.

18. The absorbent biophotonic device as defined in claim 17, wherein the polyethylene foam is a reticulated polyethylene foam.

19. The absorbent biophotonic device as defined in claim 1, wherein the absorbent biophotonic device is an absorbent biophotonic pad.

20. The absorbent biophotonic device as defined in claim 1, wherein the absorbent biophotonic device comprises a plurality of punctures connecting the photoactivatable core and the at least one absorbent liner.

36. (canceled)
